(19) United States
(12) Patent Application Publication
(10) Pub. No.: US 2010/0042174 A1
(43) Pub. Date: Feb. 18, 2010

(54) SELECTING PACING SITE OR SITES BASED ON CARDIO-PULMONARY INFORMATION

(75) Inventors: Steve Koh, South Pasadena, CA (US); Michael Yang, Thousand Oaks, CA (US); Kyungmoo Ryu, Palmdale, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/190,545

(22) Filed: Aug. 12, 2008

(51) Int. Cl. A61N 1/368

(52) U.S. Cl. 607/19

(57) ABSTRACT

An exemplary method for multi-tier pacing includes delivering single site, left ventricular pacing, sensing patient activity; comparing the sensed patient activity to a patient activity threshold and, if the sensed patient activity exceeds the patient activity threshold, then delivering multi-site, left ventricular pacing for a predetermined period of time and, after the predetermined period of time, delivering single, site left ventricular pacing. In such a method, the period of time may be determined based on cardio-pulmonary demand. Other exemplary technologies are also disclosed.

### TECHNICAL FIELD

Exemplary technologies presented herein generally relate to cardiac pacing and/or stimulation therapy. Various techniques provide for selecting pacing site or sites based on cardiopulmonary performance.

### BACKGROUND

Heart Failure (HF) is a chronic condition that affects over 5 million Americans and, according to the American Heart Association, HF accounts for more hospitalization among elderly people than any other condition. HF is not a condition in which the heart abruptly stops beating. Instead, HF refers to a dysfunction in the pumping action of the heart due to the heart's inability to contract or relax properly. It is generally experienced by patients who have suffered a heart attack or whose hearts have been damaged by other conditions which have disrupted the heart's natural electrical conduction system.

Patients with heart failure generally experience breathlessness, fatigue and fluid build-up in the arms and legs. This is caused by the heart's inability to pump enough blood to meet the body's demands. The heart can become enlarged as it attempts to compensate for the lack of pumping ability, which only worsens the condition. Typically, it is the lower chambers of the heart (ventricles) that do not beat efficiently (e.g., ventricular dyssynchrony) resulting in an increasingly ineffective heart.

The right ventricle is responsible for pumping blood to the lungs while the left ventricle is responsible for pumping blood to the rest of the body. The right atrium fills the right ventricle with deoxygenated blood while the left atrium fills the left ventricle with oxygenated blood. In a normal heart, the atria contract to fill the ventricles and then the ventricles contract in a synchronous manner to pump blood through the lungs or the body. Abnormal activation of any of the heart's four chambers reduces pumping efficiency. For example, abnormal ventricular activation can decrease ventricular filling, cause abnormal ventricular wall motion and cause mitral valve regurgitation (MR). Standard pharmacologic therapy cannot adequately resolve conduction and activation abnormalities such as left bundle branch block (LBBB) or a lengthy interventricular conduction delay (IVCD) that contribute to ventricular dyssynchrony.

Cardiac Resynchronization Therapy (CRT) provides an electrical solution to the symptoms and other difficulties brought on by HF. In many CRT systems, electrical impulses can be delivered to the tissue in the heart's two lower chambers (and typically one upper chamber). This is called biventricular pacing (BiV), and it causes the ventricles to beat in a more synchronized manner. BiV pacing improves the efficiency of each contraction of the heart and the amount of blood pumped to the body. This helps to lessen the symptoms of heart failure and, in many cases, helps to stop the progression of the disease. For patients fitted with CRT systems, clinical studies show improved quality of life (QOL), NYHA functional class, exercised tolerance, left ventricular reverse remodeling, morbidity and mortality.

For proper operation, values for a handful of CRT system parameters must be determined. In general, a clinician determines such values using information acquired from an echocardiography examination of a patient. Once the parameter values have been determined, the clinician can then program the patient's implantable CRT device. Some newer CRT systems include algorithms that can determine CRT parameter values based on cardiac electrograms measured by a patient's implantable CRT device. For example, the QUICKOPT™ algorithm (St. Jude Medical Corporation, Sylmar, Calif.) can determine atrio-ventricular interval (AV or PV) and interventricular interval (VV) in about a minute using intracardiac electrogram (IEGM) information. Noting that clinical evidence demonstrates that timing cycle optimization improves outcomes to CRT therapy and that optimal delays change over time, the QUICKOPT™ algorithm allows for efficient, frequent optimization. Further, QUICKOPT™ optimization is clinically proven to correlate with echo based techniques.

Various schemes exist for delivery of CRT (i.e., delivery of energy to a particular site or sites in the heart). For example, CRT may use a BiV only scheme or a scheme that uses a combination of RV only pacing and BiV pacing. With respect to RV pacing, clinical evidence indicates that long term RV apex pacing can be less than optimal, if not detrimental. Consequently, a need exists for CRT schemes that can reduce the amount of RV pacing. Various exemplary techniques described herein address this need as well as other needs related to cardiac pacing therapy. Some of the exemplary techniques may be use in delivering pacing therapies other than CRT.

### SUMMARY

An exemplary method for multi-tier pacing includes delivering single site, left ventricular pacing, sensing patient activity; comparing the sensed patient activity to a patient activity threshold and, if the sensed patient activity exceeds the patient activity threshold, then delivering multi-site, left ventricular pacing for a predetermined period of time and, after the predetermined period of time, delivering single, site left ventricular pacing. In such a method, the period of time may be determined based on cardio-pulmonary demand. Other exemplary technologies are also disclosed.

In general, the various methods, devices, systems, etc., described herein, and equivalents thereof, are suitable for use in a variety of pacing therapies and/or other cardiac related therapies.

### BRIEF DESCRIPTION OF THE DRAWINGS

Features and advantages of the described implementations can be more readily understood by reference to the following description taken in conjunction with the accompanying drawings.

FIG. 1 is a simplified diagram illustrating an exemplary implantable stimulation device in electrical communication with at least three leads implanted into a patient's heart and at least one other lead for delivering stimulation and/or shock therapy. Other devices with fewer leads may also be suitable in some circumstances.

FIG. 2 is a functional block diagram of an exemplary implantable stimulation device illustrating basic elements that are configured to provide cardioversion, defibrillation, pacing stimulation and/or other tissue and/or nerve stimulation. The implantable stimulation device is further configured to sense information and administer stimulation pulses responsive to such information.

FIG. 3 is a diagram of an exemplary scheme for selecting no pacing, a pacing site or pacing sites based at least in part on cardiopulmonary information.

FIG. 4 is a block diagram of an exemplary method that includes a cardiopulmonary decision tree to decide whether to select a higher energy therapy tier.

FIG. 5 is a block diagram of an exemplary method that includes a cardiopulmonary feedback loop for deciding whether to select a higher energy therapy tier.

FIG. 6 is a block diagram of an exemplary method that includes a device power supply longevity check to decide whether to select a higher energy therapy tier.

FIG. 7 is a block diagram of an exemplary cardiopulmonary module for use in a computing device and exemplary sensory inputs to the module.

FIG. 8 is a block diagram of a particular example of the exemplary module along with some inputs.

FIG. 9 is a block diagram of an exemplary method that can be optionally implemented in part by the module of FIG. 8.

FIG. 10 is a plot of events versus time as they may occur during performance of the method of FIG. 9 and result in selection of a higher energy tier.

FIG. 11 is a block diagram of an exemplary method that can be optionally implemented in part by the module of FIG. 8.

FIG. 12 is a plot of events versus time as they may occur during performance of the method of FIG. 11 and result in selection of a lower energy tier.

FIG. 13 is a block diagram of an exemplary method that includes determining a duration for delivery of a higher energy therapy tier.

FIG. 14 is a block diagram of various exemplary tiers, each shown with a lower energy tier and a higher energy tier.

### DETAILED DESCRIPTION

The following description includes the best mode presently contemplated for practicing the described implementations. This description is not to be taken in a limiting sense, but rather is made merely for the purpose of describing the general principles of the implementations. The scope of the described implementations should be ascertained with reference to the issued claims. In the description that follows, like numerals or reference designators are used at times to reference like parts or elements throughout the description.

### Overview

Exemplary techniques pertain generally to monitoring cardio-pulmonary performance and selecting no pacing, single site pacing or multi-site pacing. Such techniques can optionally reduce incidence of RV pacing, which has shown to be less than optimal or detrimental. As energy expended by an implantable device generally increases as the number of pacing sites increase, such techniques can optionally reduce energy expended by an implantable device and hence increase device longevity (e.g., time to device or battery replacement).

As described herein, therapies may be arranged as tiers and cardio-pulmonary information may be assessed in a decision tree to select a therapy tier. Cardio-pulmonary information may be cardiac specific such as heart rate or pulmonary specific such as respiration rate, noting that heart rate and respiration rate are often related through patient activity state.

An exemplary stimulation device is described below followed by an exemplary tiered scheme, an exemplary method that includes a decision tree, an exemplary method that includes a feedback loop, an exemplary method that includes a power level check, an exemplary module and some methods that may be implemented by the module, for example, in conjunction with the previously described implantable device.

### Exemplary Stimulation Device

The techniques described below are intended to be implemented in connection with any stimulation device that is configured or configurable to stimulate nerves and/or stimulate and/or shock a patient's heart.

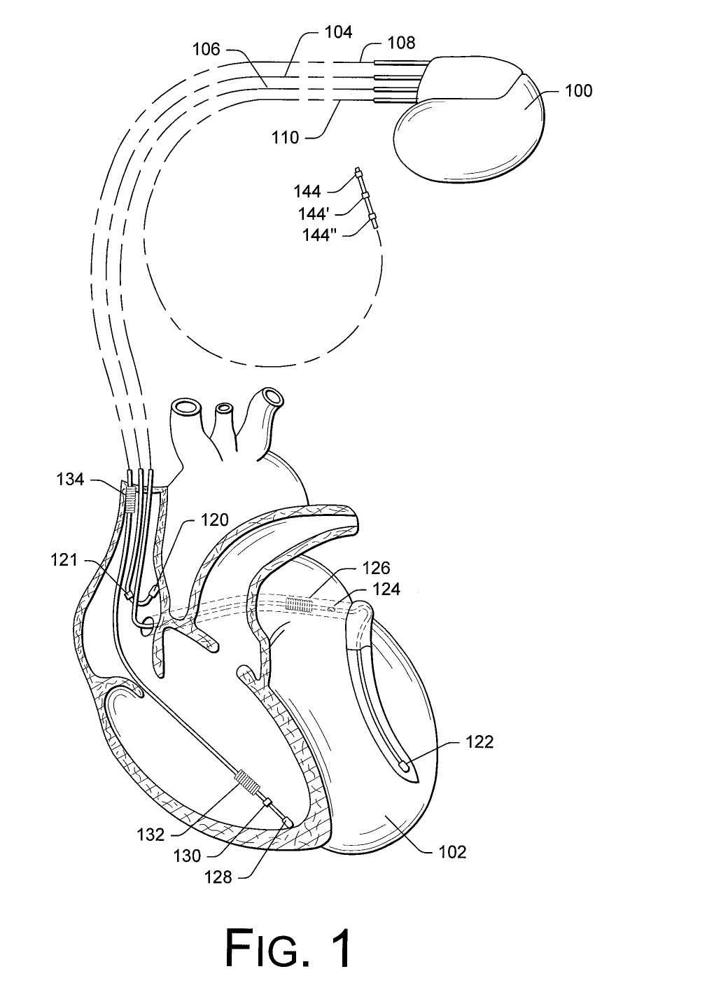

FIG. 1 shows an exemplary stimulation device 100 in electrical communication with a patient's heart 102 by way of three leads 104, 106, 108, suitable for delivering multi-chamber stimulation therapy and optionally shock therapy. In the example of FIG. 1, the device 100 includes a fourth lead 110 having, in this implementation, three electrodes 144, 144′, 144″ suitable for stimulation of tissue (e.g., myocardial tissue, muscle tissue, autonomic nerves, etc.) and/or sensing information. Lead number, lead type, electrode number, etc., can vary depending on the particular therapy or therapies to be delivered to a patient.

The right atrial lead 104 is configured to be positioned in a patient's right atrium. The implantable device 100 can use the right atrial lead 104 for delivering stimulation therapy to the right atrium. The right atrial lead 104 may also be configured to allow the device 100 to sense cardiac signals (e.g., near field atrial signals and/or far field ventricular signals). As shown in FIG. 1, the right atrial lead 104 includes an atrial tip electrode 120 (typically implanted in the patient's right atrial appendage) and an atrial ring electrode 121. The right atrial lead 104 may include one or more additional electrodes.

To sense atrial cardiac signals, ventricular cardiac signals and/or to provide chamber pacing therapy, particularly on the left side of a patient's heart, the stimulation device 100 is coupled to a coronary sinus lead 106 designed for placement in the coronary sinus and/or a tributary vein of the coronary sinus. As shown in FIG. 1, the coronary sinus lead 106 is positioned in the coronary sinus and a tributary to the coronary sinus where at least one electrode is adjacent to the left ventricle and at least one additional electrode is adjacent to the left atrium.

The coronary sinus lead 106 may be configured to receive atrial and ventricular cardiac signals and to deliver left ventricular pacing therapy. In the example of FIG. 1, the coronary sinus lead 106 includes a left ventricular tip electrode 122 suitable for delivery of left ventricular pacing therapy, a left atrial ring electrode 124 suitable for delivery of left atrial pacing therapy, and a left atrial coil electrode 126 suitable for delivery of shock therapy. For a complete description of a coronary sinus lead, the reader is directed to U.S. Pat. No. 5,466,254, “Coronary Sinus Lead with Atrial Sensing Capability” (Helland), which is incorporated herein by reference.

Stimulation device 100 is also shown in electrical communication with the patient's heart 102 by way of an implantable right ventricular lead 108 having, in this exemplary implementation, a right ventricular tip electrode 128, a right ventricular ring electrode 130, a right ventricular (RV) coil electrode 132, and an SVC coil electrode 134. Typically, the right ventricular lead 108 is transvenously inserted into the heart 102 to place the right ventricular tip electrode 128 in the right ventricular apex so that the RV coil electrode 132 will be positioned in the right ventricle and the superior vena cava (SVC) coil electrode 134 will be positioned in the SVC. Accordingly, the right ventricular lead 108 is capable of sensing or receiving cardiac signals and delivering stimulation in the form of pacing and shock therapy to the right ventricle.

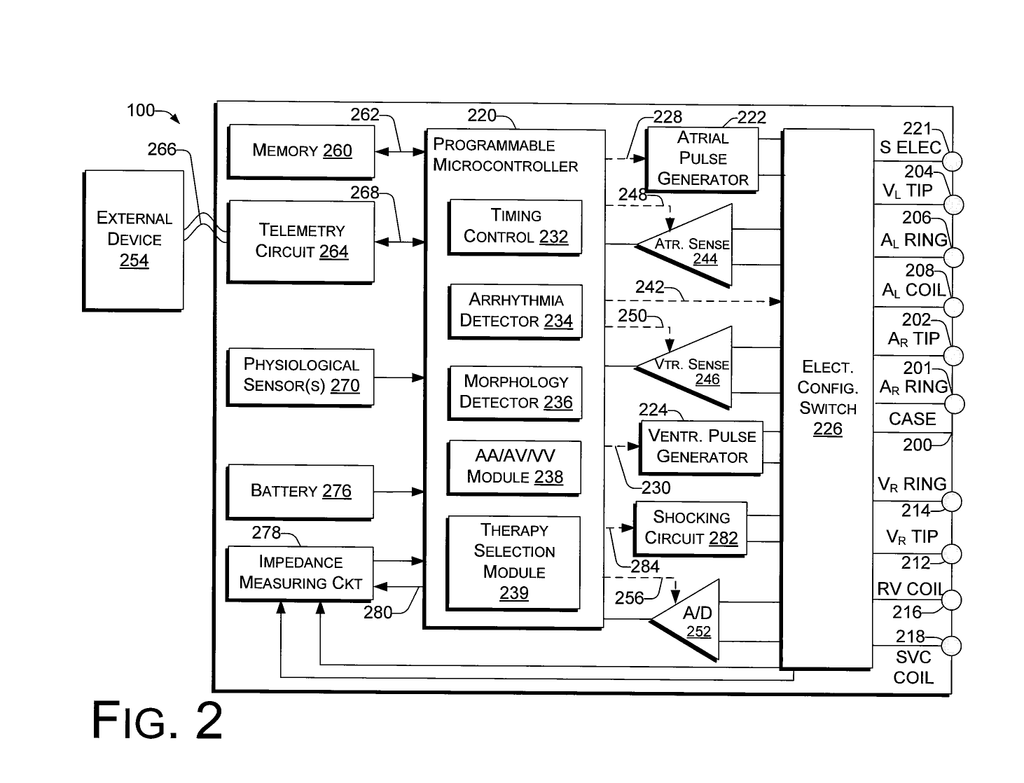

FIG. 2 shows an exemplary, simplified block diagram depicting various components of stimulation device 100. The stimulation device 100 can be capable of treating both fast and slow arrhythmias with stimulation therapy (e.g., cardioversion, defibrillation, and/or pacing stimulation). While a particular multi-chamber device is shown, it is to be appreciated and understood that this is done for illustration purposes only. Techniques and methods described below can be implemented in connection with any suitably configured or configurable stimulation device. Accordingly, one of skill in the art could readily duplicate, eliminate, or disable the appropriate circuitry in any desired combination to provide a device capable of treating the appropriate chamber(s) or regions of a patient's heart with cardioversion, defibrillation and/or pacing stimulation.

Housing 200 for stimulation device 100 is often referred to as the “can”, “case” or “case electrode”, and may be programmably selected to act as the return electrode for all “unipolar” modes. Housing 200 may further be used as a return electrode alone or in combination with one or more of the coil electrodes 126, 132 and 134 for shocking purposes. Housing 200 further includes a connector (not shown) having a plurality of terminals 201, 202, 204, 206, 208, 212, 214, 216, 218, 221 (shown schematically and, for convenience, the names of the electrodes to which they are connected are shown next to the terminals). The terminal S ELEC 221 may be used for any of a variety of purposes (e.g., sensing, nerve tissue stimulation, myocardial tissue stimulation, other muscle tissue stimulation, etc.).

To achieve right atrial sensing and/or pacing, the connector includes at least a right atrial tip terminal (AR TIP) 202 adapted for connection to the atrial tip electrode 120. A right atrial ring terminal (AR RING) 201 is also shown, which is adapted for connection to the atrial ring electrode 121. To achieve left chamber sensing, pacing and/or shocking, the connector includes at least a left ventricular tip terminal (VL TIP) 204, a left atrial ring terminal (AL RING) 206, and a left atrial shocking terminal (AL COIL) 208, which are adapted for connection to the left ventricular tip electrode 122, the left atrial ring electrode 124, and the left atrial coil electrode 126, respectively.

To support right chamber sensing, pacing and/or shocking, the connector further includes a right ventricular tip terminal (VR TIP) 212, a right ventricular ring terminal (VR RING) 214, a right ventricular shocking terminal (RV COIL) 216, and a SVC shocking terminal (SVC COIL) 218, which are adapted for connection to the right ventricular tip electrode 128, right ventricular ring electrode 130, the RV coil electrode 132, and the SVC coil electrode 134, respectively.

At the core of the stimulation device 100 is a programmable microcontroller 220 that controls the various modes of stimulation therapy. As is well known in the art, microcontroller 220 typically includes a microprocessor, or equivalent control circuitry, designed specifically for controlling the delivery of stimulation therapy, and may further include RAM or ROM memory, logic and timing circuitry, state machine circuitry, and I/O circuitry. Typically, microcontroller 220 includes the ability to process or monitor input signals (data or information) as controlled by a program code stored in a designated block of memory. The type of microcontroller is not critical to the described implementations. Rather, any suitable microcontroller 220 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. No. 4,712,555 (Thornander) and U.S. Pat. No. 4,944,298 (Sholder), all of which are incorporated by reference herein. For a more detailed description of the various timing intervals used within the stimulation device and their inter-relationship, see U.S. Pat. No. 4,788,980 (Mann et al.), also incorporated herein by reference.

FIG. 2 also shows an atrial pulse generator 222 and a ventricular pulse generator 224 that generate pacing stimulation pulses for delivery by the right atrial lead 104, the coronary sinus lead 106, and/or the right ventricular lead 108 via an electrode configuration switch 226. It is understood that in order to provide stimulation therapy in each of the four chambers of the heart (or to nerves or other tissue) the atrial and ventricular pulse generators, 222 and 224, may include dedicated, independent pulse generators, multiplexed pulse generators, or shared pulse generators. The pulse generators 222 and 224 are controlled by the microcontroller 220 via appropriate control signals 228 and 230, respectively, to trigger or inhibit the stimulation pulses.

Microcontroller 220 further includes timing control circuitry 232 to control the timing of the stimulation pulses (e.g., pacing rate, atrio-ventricular (e.g., AV) delay, atrial interconduction (AA) delay, or ventricular interconduction (VV) delay, etc.) as well as to keep track of the timing of refractory periods, blanking intervals, noise detection windows, evoked response windows, alert intervals, marker channel timing, etc., which is well known in the art.

Microcontroller 220 further includes an arrhythmia detector 234, a morphology detector 236, and optionally an orthostatic compensator and a minute ventilation (MV) response module; the latter two are not shown in FIG. 2. These components can be utilized by the stimulation device 100 for determining desirable times to administer various therapies, including those to reduce the effects of orthostatic hypotension. The aforementioned components may be implemented in hardware as part of the microcontroller 220, or as software/firmware instructions programmed into the device and executed on the microcontroller 220 during certain modes of operation.

Microcontroller 220 further includes an AA delay, AV delay and/or W delay module 238 for performing a variety of tasks related to M delay, AV delay and/or VV delay. The module 238 may optionally implement the aforementioned QuickOpt™ technique for determining one or more pacing timing parameters based on intracardiac electrogram information. The component 238 can be utilized by the stimulation device 100 for determining desirable times to administer various therapies, including, but not limited to, ventricular stimulation therapy, bi-ventricular stimulation therapy, resynchronization therapy, atrial stimulation therapy, etc. The AA/AV/VV module 238 may be implemented in hardware as part of the microcontroller 220, or as software/firmware instructions programmed into the device and executed on the microcontroller 220 during certain modes of operation. Of course, such a module may be limited to one or more of the particular functions of AA delay, AV delay and/or VV delay. Such a module may include other capabilities related to other functions that may be germane to the delays. Such a module may help make determinations as to fusion.

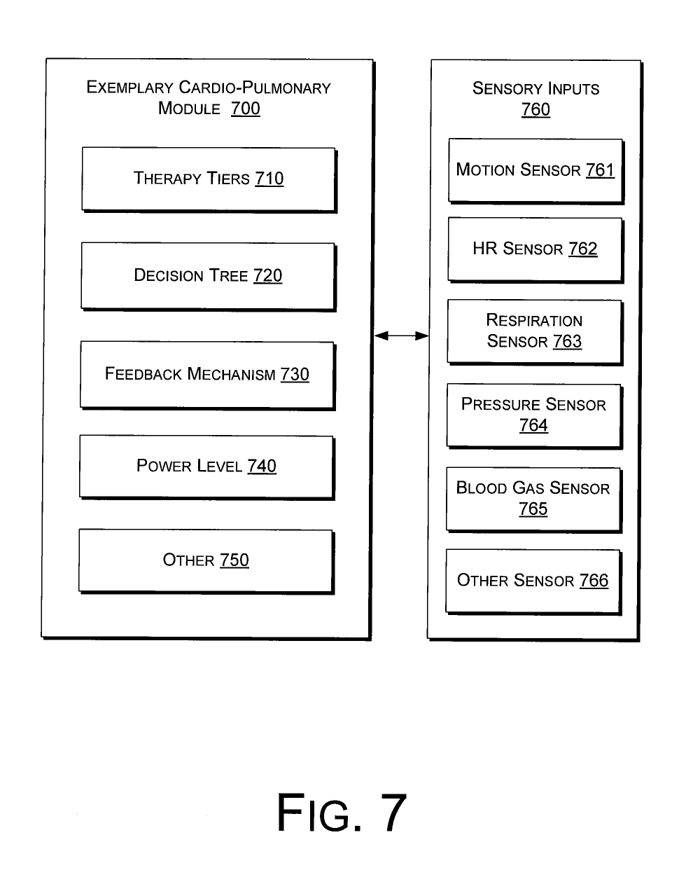

The microcontroller 220 of FIG. 2 also includes a therapy selection module activity module 239. This module may include control logic for one or more therapy selection related features. For example, the module 239 may include an algorithm for assessing patient activity information, calling for respiratory information, selecting one or more pacing sites based on sensed information, etc. Such algorithms are described in more detail with respect to the figures. Also, the module 239 may include at least some of the features of module 700 of FIG. 7. The module 239 may be implemented in hardware as part of the microcontroller 220, or as software/firmware instructions programmed into the device and executed on the microcontroller 220 during certain modes of operation. The module 239 may act cooperatively with the AA/AV/VV module 238.

The electronic configuration switch 226 includes a plurality of switches for connecting the desired electrodes to the appropriate I/O circuits, thereby providing complete electrode programmability. Accordingly, switch 226, in response to a control signal 242 from the microcontroller 220, determines the polarity of the stimulation pulses (e.g., unipolar, bipolar, combipolar, etc.) by selectively closing the appropriate combination of switches (not shown) as is known in the art.

Atrial sensing circuits 244 and ventricular sensing circuits 246 may also be selectively coupled to the right atrial lead 104, coronary sinus lead 106, and the right ventricular lead 108, through the switch 226 for detecting the presence of cardiac activity in each of the four chambers of the heart. Accordingly, the atrial (ATR. SENSE) and ventricular (VTR. SENSE) sensing circuits, 244 and 246, may include dedicated sense amplifiers, multiplexed amplifiers, or shared amplifiers. Switch 226 determines the “sensing polarity” of the cardiac signal by selectively closing the appropriate switches, as is also known in the art. In this way, the clinician may program the sensing polarity independent of the stimulation polarity. The sensing circuits (e.g., 244 and 246) are optionally capable of obtaining information indicative of tissue capture.

Each sensing circuit 244 and 246 preferably employs one or more low power, precision amplifiers with programmable gain and/or automatic gain control, bandpass filtering, and a threshold detection circuit, as known in the art, to selectively sense the cardiac signal of interest. The automatic gain control enables the device 100 to deal effectively with the difficult problem of sensing the low amplitude signal characteristics of atrial or ventricular fibrillation.

The outputs of the atrial and ventricular sensing circuits 244 and 246 are connected to the microcontroller 220, which, in turn, is able to trigger or inhibit the atrial and ventricular pulse generators 222 and 224, respectively, in a demand fashion in response to the absence or presence of cardiac activity in the appropriate chambers of the heart. Furthermore, as described herein, the microcontroller 220 is also capable of analyzing information output from the sensing circuits 244 and 246 and/or the data acquisition system 252 to determine or detect whether and to what degree tissue capture has occurred and to program a pulse, or pulses, in response to such determinations. The sensing circuits 244 and 246, in turn, receive control signals over signal lines 248 and 250 from the microcontroller 220 for purposes of controlling the gain, threshold, polarization charge removal circuitry (not shown), and the timing of any blocking circuitry (not shown) coupled to the inputs of the sensing circuits, 244 and 246, as is known in the art.

For arrhythmia detection, the device 100 utilizes the atrial and ventricular sensing circuits, 244 and 246, to sense cardiac signals to determine whether a rhythm is physiologic or pathologic. In reference to arrhythmias, as used herein, “sensing” is reserved for the noting of an electrical signal or obtaining data (information), and “detection” is the processing (analysis) of these sensed signals and noting the presence of an arrhythmia. In some instances, detection or detecting includes sensing and in some instances sensing of a particular signal alone is sufficient for detection (e.g., presence/absence, etc.).

The timing intervals between sensed events (e.g., P-waves, R-waves, and depolarization signals associated with fibrillation) are then classified by the arrhythmia detector 234 of the microcontroller 220 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Cardiac signals are also applied to inputs of an analog-to-digital (A/D) data acquisition system 252. The data acquisition system 252 is configured to acquire intracardiac electrogram (IEGM) signals, convert the raw analog data into a digital signal, and store the digital signals for later processing and/or telemetric transmission to an external device 254. The data acquisition system 252 is coupled to the right atrial lead 104, the coronary sinus lead 106, the right ventricular lead 108 and/or the optional lead 110 through the switch 226 to sample cardiac signals across any pair of desired electrodes.

The microcontroller 220 is further coupled to a memory 260 by a suitable data/address bus 262, wherein the programmable operating parameters used by the microcontroller 220 are stored and modified, as required, in order to customize the operation of the stimulation device 100 to suit the needs of a particular patient. Such operating parameters define, for example, pacing pulse amplitude, pulse duration, electrode polarity, rate, sensitivity, automatic features, arrhythmia detection criteria, and the amplitude, waveshape, number of pulses, and vector of each shocking pulse to be delivered to the patient's heart 102 within each respective tier of therapy. The device 100 typically includes the ability to sense and store a relatively large amount of data (e.g., from the data acquisition system 252), which data may then be used for subsequent analysis, for example, to guide the programming of the device.

Various types of information (e.g., parameter values, modes, etc.) for the implantable device 100 may be non-invasively programmed into the memory 260 through a telemetry circuit 264 in telemetric communication via communication link 266 with the external device 254, such as a programmer, transtelephonic transceiver, or a diagnostic system analyzer. The microcontroller 220 activates the telemetry circuit 264 with a control signal 268. The telemetry circuit 264 allows intracardiac electrograms (IEGMs) and status information relating to the operation of the device 100 (as contained in the microcontroller 220 or memory 260) to be sent to the external device 254 through an established communication link 266.

The stimulation device 100 can include one or more physiological sensors 270. For example, the device 100 may include a “rate-responsive” to adjust pacing stimulation rate according to the exercise state of the patient. The device 100 may include a physiological sensor 270 to detect changes in cardiac output (see, e.g., U.S. Pat. No. 6,314,323, entitled “Heart stimulator determining cardiac output, by measuring the systolic pressure, for controlling the stimulation”, to Ekwall, issued Nov. 6, 2001, which discusses a pressure sensor adapted to sense pressure in a right ventricle and to generate an electrical pressure signal corresponding to the sensed pressure, an integrator supplied with the pressure signal which integrates the pressure signal between a start time and a stop time to produce an integration result that corresponds to cardiac output), to detect changes in the physiological condition of the heart, or to detect diurnal changes in activity (e.g., detecting sleep and wake states). With respect to pressure sensors for left atrial pressure, an implantable sensor (marketed by St. Jude Medical, Sylmar, Calif.) can be positioned via the atrial septum to measure left atrial pressures. Left atrial pressure information can help detect and manage symptoms associated with progressive heart failure as increased pressure in the left atrium is a predictor of pulmonary congestion, which is the leading cause of hospitalization for congestive heart failure patients. The microcontroller 220 may be programmed to respond to sensed information by adjusting one or more therapy parameters (such as rate, AA delay, AV delay, VV delay, etc.).

While the block 270 is shown as being included within the stimulation device 100, it is to be understood that a physiologic sensor may also be external to the stimulation device 100, yet still be implanted within or carried by the patient. Examples of physiologic sensors that may be implemented in device 100 include known sensors that, for example, sense respiration rate, pH of blood, blood gas, ventricular gradient, cardiac output, preload, afterload, contractility, hemodynamics, pressure, and so forth. Another sensor that may be used is one that detects activity variance. For example, an activity sensor may be monitored diurnally to detect the low variance in a measurement as corresponding to a patient's sleep state. For a complete description of an activity variance sensor, the reader is directed to U.S. Pat. No. 5,476,483 (Bornzin et al.), issued Dec. 19, 1995, which patent is hereby incorporated by reference.

With respect to sensing blood gas concentrations, oximeter sensors are disclosed in U.S. patent application Ser. No. 11/231,555, entitled “Implantable multi-wavelength oximeter sensor”, filed Sep. 20, 2005 and U.S. patent application Ser. No. 11/282,198, entitled “Implantable self-calibrating optical sensors”, filed Nov. 11, 2005, which are incorporated by reference herein. An oximeter sensor can provide a signal indicative of blood oxygen level, which, in turn, can be used to assess cardiopulmonary demand and/or performance. For example, as demand increases (e.g., due to patient activity), a drop in blood oxygen concentration occurs if cardio-pulmonary performance can not meet the demand.

The one or more physiological sensors 270 optionally include sensors for detecting minute ventilation and/or sensors for detecting movement and/or position. A minute ventilation (MV) sensor senses minute ventilation, which is defined as the total volume of air that moves in and out of a patient's lungs in a minute. A movement and/or position sensor may rely on spring loaded moving mass(es) that responds to movement (e.g., acceleration) and/or patient position (e.g., angle with respect to acceleration of gravity). Micro-electromechanical system (MEMS) accelerometers are available on a single monolithic IC that consumes low power and include mechanisms aligned three axes with signal conditioned voltage outputs. Such a sensor can measure the static acceleration of gravity in tilt-sensing applications (e.g., patient tilt), as well as dynamic acceleration resulting from motion, shock, or vibration (e.g., patient movement).

Signals generated by a sensor can be passed to the microcontroller 220 for analysis in determining whether to adjust one or more settings (e.g., AV delay, VV delay, pacing rate, etc.). For example, the microcontroller 220 may monitor a sensor's signal for indications of a patient's position and/or activity status, such as whether the patient is climbing upstairs or descending downstairs or whether the patient is sitting up after lying down, and adjust one or more settings to accommodate the patient's activity. As described in more detail further below, an exemplary implantable device can select an appropriate therapy based on any of a variety of sensed information. Such a selection may include selecting a therapy from a no pacing therapy, a single pacing site therapy and a multiple pacing site therapy.

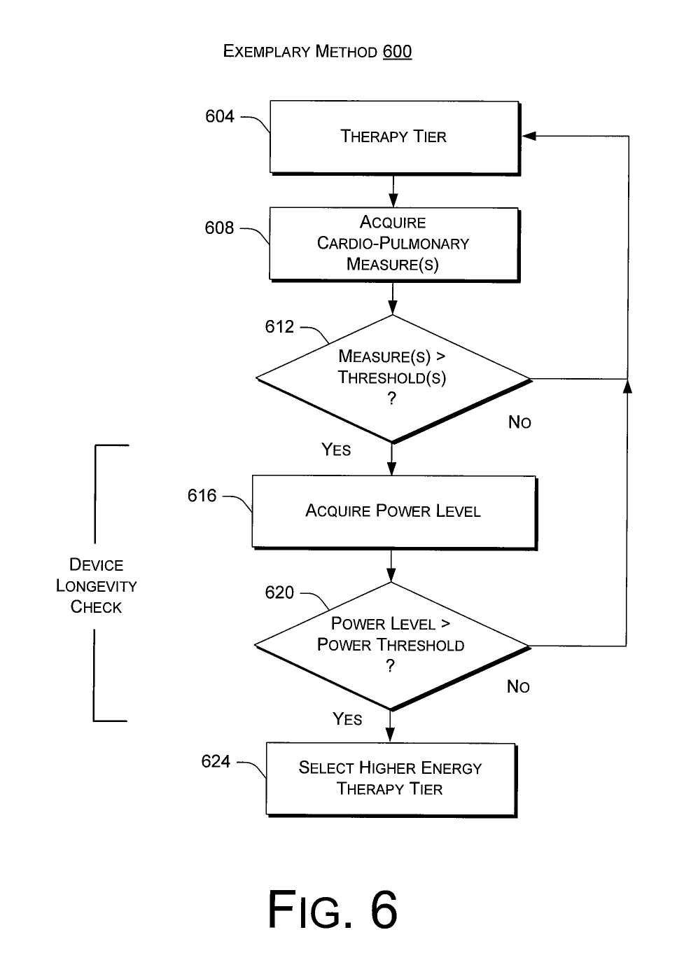

The stimulation device 100 additionally includes a battery 276 that provides operating power to all of the circuits shown in FIG. 2. For the stimulation device 100, which employs shocking therapy, the battery 276 is capable of operating at low current drains for long periods of time (e.g., preferably less than 10 μA), and is capable of providing high-current pulses (for capacitor charging) when the patient requires a shock pulse (e.g., preferably, in excess of 2 A, at voltages above 2 V, for periods of 10 seconds or more). The battery 276 also desirably has a predictable discharge characteristic so that elective replacement time can be detected. As described herein, the therapy selection module 239 can receive information germane to battery level or more generally power level. Such information may be a direct reading of a power indicator or may be a schedule that indicates time to replacement. In the latter instance, a care provider may optionally update a time to replacement parameter(s) after communicating with the implantable device (e.g., as in a routine follow-up visit). As described below (see, e.g., the method 600 of FIG. 6), power level information may be used in deciding whether to select a higher energy level tier.

The stimulation device 100 can further include magnet detection circuitry (not shown), coupled to the microcontroller 220, to detect when a magnet is placed over the stimulation device 100. A magnet may be used by a clinician to perform various test functions of the stimulation device 100 and/or to signal the microcontroller 220 that the external programmer 254 is in place to receive or transmit data to the microcontroller 220 through the telemetry circuits 264.

The stimulation device 100 further includes an impedance measuring circuit 278 that is enabled by the microcontroller 220 via a control signal 280. The known uses for an impedance measuring circuit 278 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring edema; measuring intrathoracic impedance for determining shock thresholds; detecting when the device has been implanted; measuring stroke volume; and detecting the opening of heart valves, etc. With respect to respiration or minute ventilation, spaced electrodes (e.g., can plus a lead-based electrode positioned in the heart) can be used to measure intrathoracic impedance as the distance and/or material properties of the conducting media can change during respiration. For example, as the lungs expand during inspiration, the conduction path between a can electrode (e.g., positioned in a pectoral pocket) and an intracardiac electrode can change as well as the nature of the conducting media along the path (e.g., body tissue, fluid, etc.). The impedance measuring circuit 278 is advantageously coupled to the switch 226 so that any desired electrode configuration may be used.

In the case where the stimulation device 100 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 220 further controls a shocking circuit 282 by way of a control signal 284. The shocking circuit 282 generates shocking pulses of low (e.g., up to approximately 0.5 J), moderate (e.g., approximately 0.5 J to approximately 10 J), or high energy (e.g., approximately 11 J to approximately 40 J), as controlled by the microcontroller 220. Such shocking pulses are applied to the patient's heart 102 through at least two shocking electrodes, and as shown in this embodiment, selected from the left atrial coil electrode 126, the RV coil electrode 132, and/or the SVC coil electrode 134. As noted above, the housing 200 may act as an active electrode in combination with the RV electrode 132, or as part of a split electrical vector using the SVC coil electrode 134 or the left atrial coil electrode 126 (i.e., using the RV electrode as a common electrode). Other exemplary devices may include one or more other coil electrodes or suitable shock electrodes (e.g., a LV coil, etc.).

Cardioversion level shocks are generally considered to be of low to moderate energy level (where possible, so as to minimize pain felt by the patient), and/or synchronized with an R-wave and/or pertaining to the treatment of tachycardia. Defibrillation shocks are generally of moderate to high energy level (i.e., corresponding to thresholds in the range of approximately 5 J to approximately 40 J), delivered asynchronously (since R-waves may be too disorganized), and pertaining exclusively to the treatment of fibrillation. Accordingly, the microcontroller 220 is capable of controlling the synchronous or asynchronous delivery of the shocking pulses.

### Exemplary Scheme

Evidence from a recent clinical study indicates that LV only pacing provides essentially equivalent hemodynamic benefits to conventional BiV pacing when the patients are in a low active condition (Bordachar et al., “Echocardiographic Assessment During Exercise of Heart Failure Patients With Cardiac Resynchronization Therapy”, Am J Cardiol 2006; 97:1622-1625). However, under a relatively active condition, CRT patients may benefit more from BiV pacing than LV only pacing.

According to Bordachar et al., LV only pacing prolonged the systolic period to the detriment of diastolic filling, with negative hemodynamic consequence during exercise. This prompted a separate optimization of VV interval at rest and during exercise, which led to significant hemodynamic improvement. However, the optimization resulted in a VV interval that differed from at rest to exercise in more than half of enrolled patients. Consequently, if BiV is delivered at rest and during exercise, the VV interval should be adjusted, for example, using the aforementioned QuickOpt™ technique. Otherwise, a patient may have (i) a VV interval suitable for rest and unsuitable for an active state; (ii) a VV interval suitable for an active state and unsuitable for rest; or (iii) a VV interval somewhat suitable for rest and somewhat suitable for an active state.

As described herein, an exemplary method can switch from a single ventricle pacing therapy to a BiV pacing therapy based at least in part on patient activity information. More generally, such a method can be described with respect to selection of pacing site or pacing sites. For example, a LV only pacing therapy may be single site or multi-site in the LV; whereas, a BiV pacing therapy must include at least one RV site and at least one LV site. In general, the power expended to activate the heart increases as the number of sites increase. In essence, a capture threshold exists at each site and the energy delivered at any given site should be sufficient to cause an evoked response. Hence, LV only pacing during rest and BiV pacing during activity can extend the life of an implantable CRT device that would otherwise call for BiV during both patient rest and active states.

As for the choice of LV only versus RV only pacing during a rest state, it is known from the DAVID study (which pursued the hypothesis that dual-chamber ICDs provide improved patient prognosis and reduced health care costs as opposed to single-chamber ICDs) that long term RV apex pacing can be less than optimal, if not detrimental. Thus, various exemplary methods, where appropriate, use no pacing or LV only pacing during patient rest. Such methods aim to minimize RV stimulation related side effects for CRT patients. Such methods may also conserve power at the same time by appropriately determining when to select LV only pacing and when to select BiV pacing.

An exemplary implantable device includes an RV lead and an LV lead for delivery of BiV or LVP based on activity. When activity is low (common for HF patients), the device selects LV only pacing. When an activity sensor reading increases such as in response to a patient climbing stairs or walking, the device can select BiV. As described in more detail below, such a selection or switching may occur after considering other factors such as breathing and heart rate (e.g., as well as activity information).

With respect to respiration, increased breathing rate is usually triggered by chemoreceptor detection of hydrogen ions [H⁺] as surrogate for blood gas CO₂ concentration. As described herein, a trigger to switching to a more optimal setting can use any of a variety of physiological clues indicative of an increase in metabolic demand. For example, one or more of the following physiological measures may be used: blood flow, pressure, tissue stretch, QTc, T wave, FFT of an electrogram, pH or CO₂, O₂ saturation, impedance, intra-ventricular delay, interventricular delay or CO.

For HF patients, it is essential to boost pumping function to improve O₂—CO₂ gas exchange in the patient's lungs to promote well-being as well as lessening chance of exercise induced ischemia (low oxygen, poor circulation, etc.).

For some patients, LV only pacing may be improved via pacing at multiple LV sites. Multisite LV pacing can also activate the LV cardiac muscle in a sequential manner and thereby increase contractility and decrease mechanical dysynchrony. As most HF patients have LV damage or disease, for this patient population, it is possible to implant only a LV lead (i.e., to not implant an RV lead). Where multiple sites are used in a single ventricle for pacing a single ventricle (i.e., RV only or LV only, either of which may include atrial pacing), such therapies are referred to as RV based CRT or LV based CRT, noting that LV based CRT will be more commonly implemented.

Exemplary techniques that eliminate the need for an RV lead provide advantages as to improved reliability, cost, and short implant time. Similarly, for RV based CRT, some patients may forego implantation of a LV lead.

Various exemplary techniques can switch/unswitch between a single site pacing in a chamber(s) (LV, RV, or BiV) and multisite pacing in a chamber(s) (LV, RV, or BiV) using information from one or more physiological sensors (e.g., including impedance, electrogram, etc., information). Various studies further indicate that multisite LV and/or multisite RV pacing improve CRT performance.

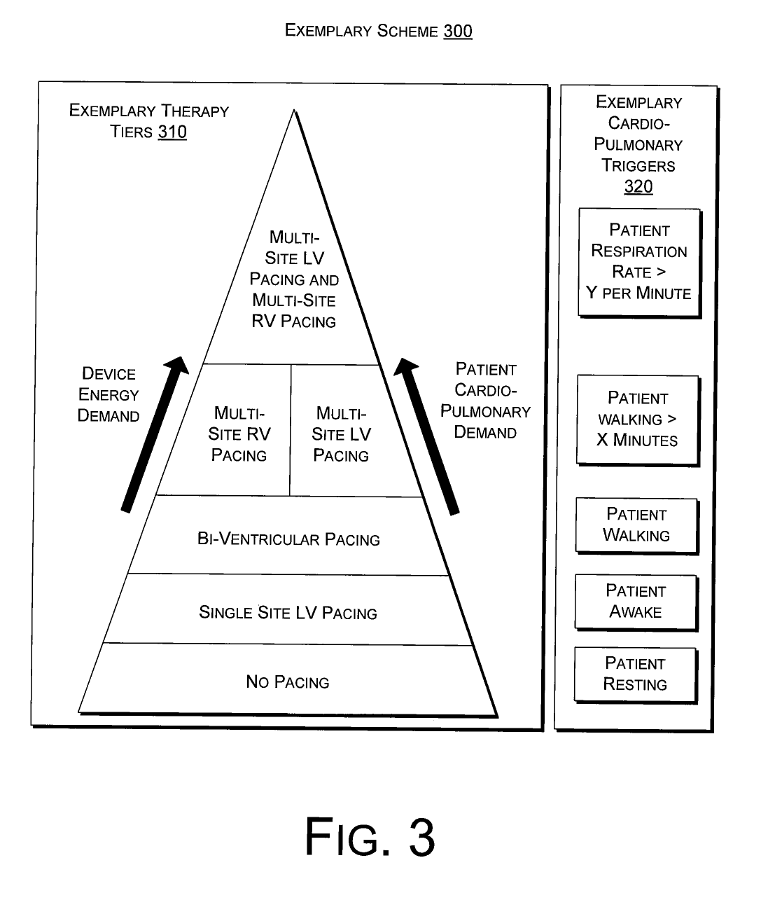

FIG. 3 shows an exemplary scheme 300 for selecting an appropriate therapy tier from exemplary therapy tiers 310. The tiers 310 include a base level no pacing tier, a single site LV pacing tier, a BiV pacing tier, a multi-site RV pacing tier, a multi-site LV pacing tier and a multi-site LV and multi-site RV pacing tier. In general, the higher tiers consume more energy yet may more effectively assist a patient in meeting cardio-pulmonary demand.

A tier of the tiers 310 may be selected by one or more triggers. Exemplary cardiopulmonary triggers 320 include patient resting (e.g., to trigger lower tier), patient awake (e.g., to trigger an algorithm to acquire or sense more information), patient walking (e.g., to trigger a BiV tier), patient walking for greater than a certain number of minutes (e.g., to trigger multi-site pacing tier), patient respiration rate greater than a certain rate (e.g., to trigger a multi-site BiV pacing tier).

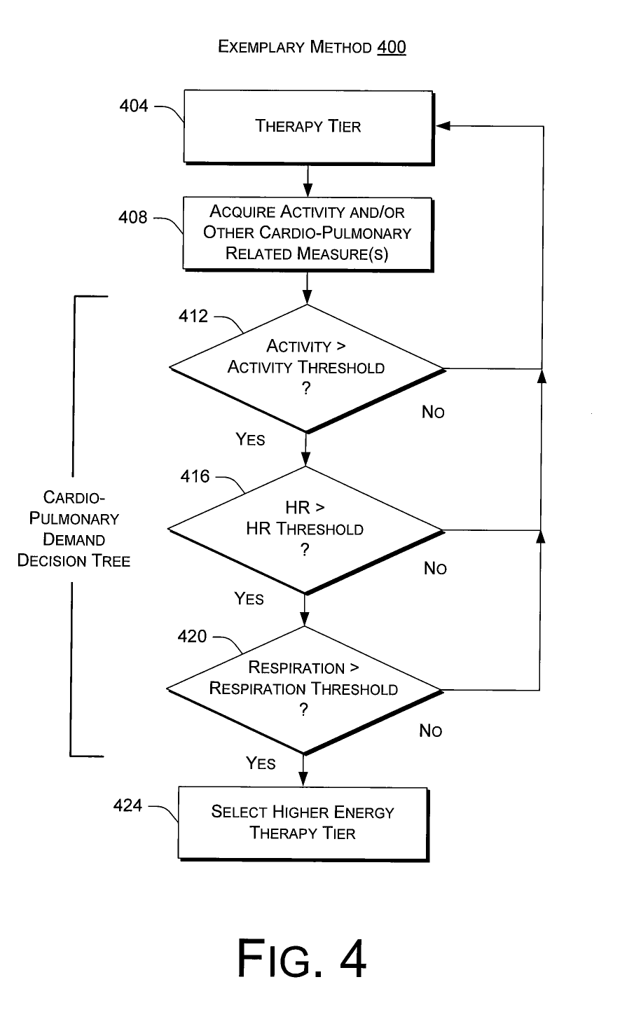

FIG. 4 shows an exemplary method 400 for selecting a therapy tier based at least in part on cardio-pulmonary information. The method 400 commences in a therapy tier block 404. An acquisition block 408 follows to acquire activity and/or one or more other cardiopulmonary related measures. Next, the method 400 enters a cardio-pulmonary demand decision tree that includes three decision block 412, 416 and 420. The decision block 412 decides if the activity exceeds an activity threshold, which may be a programmable value, a fixed value or a value adjusted by an algorithm of an implantable device. If the decision block 412 decides that the activity does not exceed the activity threshold, then the method 400 continues to operate in the therapy tier per block 404. However, if the activity exceeds the activity threshold, then the method 400 continues to another decision block 416.

The decision block 416 decides if heart rate (e.g., programmed or intrinsic) exceeds a heart rate threshold, which may be a programmable value, a fixed value or a value adjusted by an algorithm of an implantable device. If the decision block 416 decides that the heart rate does not exceed the heart rate threshold, then the method 400 continues to operate in the therapy tier per block 404. However, if the activity exceeds the heart rate threshold, then the method 400 continues to another decision block 420.

The decision block 420 decides if respiration rate exceeds a respiration rate threshold, which may be a programmable value, a fixed value or a value adjusted by an algorithm of an implantable device. If the decision block 420 decides that the respiration rate does not exceed the respiration rate threshold, then the method 400 continues to operate in the therapy tier per block 404. However, if the activity exceeds the respiration rate threshold, then the method 400 continues to selection block 424 where the method 400 selects a higher energy therapy tier in an effort to meet the cardio-pulmonary demand of the patient.

With respect to the cardio-pulmonary decision tree, the tree starts with activity, then progresses to heart rate and finally respiration rate. While the order may differ, the particular order has physiologic significance as activity is a base indicator, followed by heart rate and then respiration rate as to cardio-pulmonary demand or how well a patient is meeting his or her demand. In essence, respiration rate does not typically increase until heart rate has and likewise heart rate usually increases only after some indicator of activity.

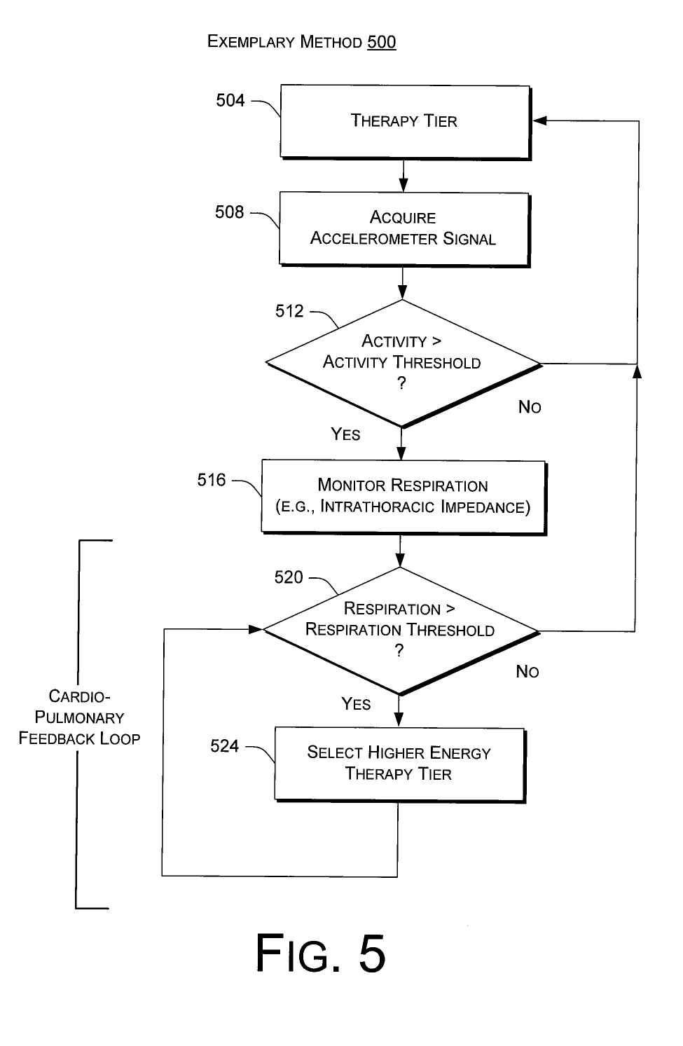

FIG. 5 shows an exemplary method 500 that includes a feedback loop. The method 500 commences in a therapy tier 504 whereby an implantable device delivers a therapy such as one of the therapies of FIG. 3, with exception of the highest level therapy. Next, in an acquisition block 508, the method 500 acquires an accelerometer signal indicative of patient activity. A decision block 512 follows that decides whether the patient activity exceeds an activity threshold. If the activity does not exceed the threshold, then it is likely that the patient is in a particular activity state suited to the therapy tier 504. However, if the activity exceeds the threshold, then the method 500 progresses to a monitoring block 516 to monitor patient respiration, for example, using intrathoracic impedance or another signal.

In the example of FIG. 5, the method 500 monitors respiration only when activity exceeds a threshold. In other examples, such a method may monitor respiration continuously or in another manner.

Once the method 500 acquires sufficient information as to patient respiration per monitoring block 516, the method 500 proceeds to a decision block 520 that decides whether the respiration information (e.g., a respiration measure such as rate, tidal volume, etc.) exceeds a respiration threshold. If the decision block 520 decides that the respiration does not exceed the threshold, then the method 500 continues to delivery therapy according to the tier of block 504. However, if the respiration exceeds the threshold, then the method 500 enters a selection block 524 to select an appropriate therapy (e.g., a higher energy therapy) in an effort to meet the patient's cardio-pulmonary demand.

Monitoring of respiration per block 516 continues after the selection block 524 whereby the decision block 520 forms a feedback loop that can cause the method 500 to revert back to the therapy tier at block 504 in the instance that the respiration no longer exceeds the respiration threshold (e.g., the respiration rate no longer exceeds a respiration rate threshold). In such a manner, the amount of time spent at the higher energy therapy tier is limited by some characteristic or characteristics of the patient's respiration. The feedback loop can act to ensure that a higher energy therapy tier is not implemented for a period of time longer than necessary, which, in turn, can conserve energy of an implantable device.

FIG. 6 shows an exemplary method 600 that includes a power level check, which may help in deciding therapy options as well as planning future of an implantable device (e.g., limit therapy options, schedule for replacement of battery, schedule for replacement of device, etc.).

The method 600 of FIG. 6 commences in a therapy tier block 604. An acquisition block 608 follows that acquires one or more cardio-pulmonary measures. A decision block 612 decides whether one or more of the measures, as appropriate, exceeds a correspond threshold; noting that the manner of acquisition per block 608 and comparison per decision block 612 may differ from that shown in FIG. 6.

In the instance that the decision block 612 decides that a patient's cardiopulmonary demand (per the one or more acquired measures) has increased, the method 600 continues at an acquisition block 616 to acquire a power level or other power related information. Otherwise, the method 600 continues to deliver therapy at the tier of block 604.

Once the method 600 acquires power information per block 616, a decision block 620 decides if the power level exceeds a power level threshold. If the decision block 620 decides that the power is too low, then it may be inappropriate or otherwise inadvisable to implement a therapy that will consume more energy of an implantable device's limited supply. Hence, the method 600 continues at the therapy block 604, where it may be noted (e.g., a flag set) that power is less than a certain threshold, which may cause the device to enter an energy conservation mode that limits data acquisition or other activities.

In the instance that the decision block 620 decides that the power level exceeds the threshold, then the method 600 enters a selection block 624 that selects a higher energy therapy tier in an effort to meet the patient's cardio-pulmonary demand as indicated by the one or more measures of block 608.

FIG. 7 shows an exemplary cardio-pulmonary module 700 and some sensory inputs 760. Referring to the device 100 of FIGS. 1 and 2, the module 239 optionally includes one or more features of the module 700 and the device 100 optionally includes one or more sensors to provide one or more of the sensory inputs 760.

In the example of FIG. 7, the module 700 includes a therapy tiers component 710 (see, e.g., FIG. 3), a decision tree component 720 (see, e.g., FIG. 4), a feedback mechanism component 730 (see, e.g., FIG. 5), a power level component 740 (see, e.g., FIG. 6) and optionally one or more other components 750. The module 700 may be in the form of software instructions (e.g., processor executable instructions) and/or hardware. The module 700 may be considered control logic for controlling an implantable device, particularly for selecting a therapy based at least in part on sensory information related to cardiopulmonary function of a patient.

As indicated in FIG. 7, the module 700 may receive information as sensory input, where sensory inputs 760 include a information from a motion sensor 761, a heart rate sensor 762, a respiration sensor 763, a pressure sensor 764, a blood gas sensor 765 and/or one or more other sensors 766 (e.g., including cardiac electrogram, neuro-electrogram, non-myocardial muscle activity, etc.).

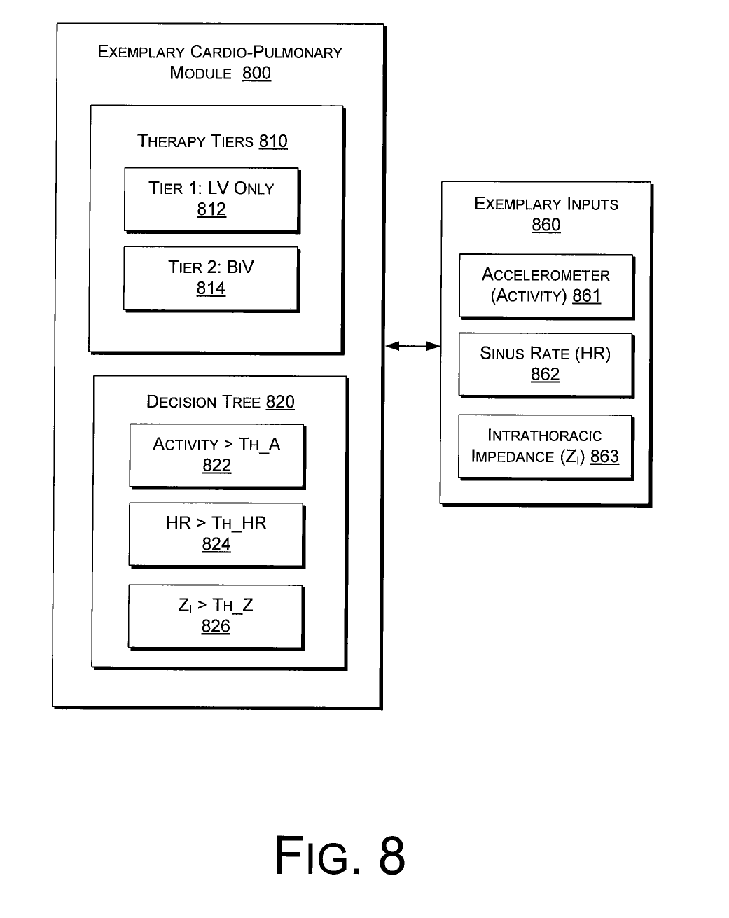

FIG. 8 shows an exemplary module 800 and exemplary inputs 860. the module 800 includes a therapy tiers component 810 and a decision tree component 820. The therapy tiers component 810 includes tier 1 LV only pacing 812 and tier 2 BiV pacing 814. The decision tree 820 includes an activity block 822 with an activity threshold Th_A, a heart rate block 824 with a heart rate threshold Th_HR and an intrathoracic impedance block 826 with an intrathoracic impedance related threshold Th_Z. As already explained, the intrathoracic impedance can provide respiratory information.

The various decision tree components 822, 824 and 826 require input information, such as provided by the exemplary inputs 860. The inputs 860 include an accelerometer 861 to provide activity information, a heart rate sensor 862 (e.g., cardiac electrogram or other) to provide heart rate information and an intrathoracic impedance sensor 863 to provide respiratory information.

In the example of FIG. 8, the inputs 860 allow the module 800 to appropriately select tier 1 therapy 812 or tier 2 therapy 814 according to the decision tree 820. As the decision tree 820 pertains to cardio-pulmonary information, it may be referred to at times as a cardio-pulmonary decision tree.

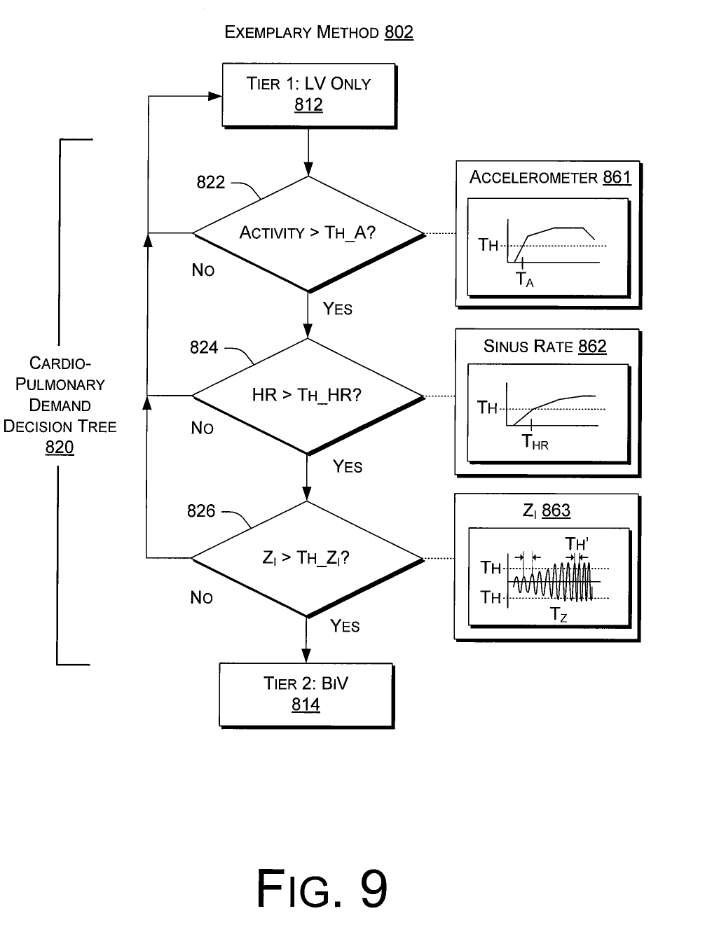

FIG. 9 shows an exemplary method 802 that can be implemented using the module 800 of FIG. 8. To provide a correspondence between the module 800 and the method 802, various block of the method 802 bear the reference numerals of their corresponding module components.

The method 802 commences in tier 1 therapy 812. The method 802 acquires information from the accelerometer 861. A corresponding plot shows activity versus time where activity exceeds a threshold at a time T_{A}. The information is processed by the decision block 822 of the decision tree 820. If the time of the accelerometer reading is less than T_{A}, then the method 802 will continue at delivery of tier 1 therapy 812. If the time is at or exceeding T_{A}, then the method 802 will acquire information as to sinus rate 862. A corresponding plot shows heart rate versus time where heart rate exceeds a threshold at a time T_{HR}. The information is processed by the decision block 824 of the decision tree 820. If the time of the sinus rate reading is less than T_{HR}, then the method 802 will continue at delivery of tier 1 therapy 812. If the time is at or exceeding T_{HR}, then the method 802 will acquire information as to respiration 863.

For respiration, a corresponding plot shows variation in impedance signal versus time where the signal exceeds a threshold at a time T_{Z}. A threshold may be for amplitude of impedance signal (which can indicate tidal volume) or peak-to-peak time difference (which can indicate respiration rate). The information is processed by the decision block 826 of the decision tree 820. If the time of the impedance reading is less than T_{Z}, then the method 802 will continue delivery of tier 1 therapy 812. Otherwise, the method 802 will select tier 2 BiV therapy 814.

Referring again to the module 800 of FIG. 8, this module may include one or more other components for delivery of therapy to improve cardio-pulmonary performance of a patient. For example, in some patients, diaphragmatic stimulation may be an option. The module 800 may select a therapy tier (e.g., Tier 1 812 or Tier 2 814) and augment the selected therapy with diaphragmatic stimulation to increase or to otherwise control respiration. In turn, input responsive to diaphragmatic stimulation may be received from an intrathoracic impedance sensor 863. Further, a call for delivery of diaphragmatic stimulation may alter the decision tree 820. Specifically, the impedance threshold Th_Z may be adjusted in impedance block 826. Such an adjustment (e.g., setting a high value for Th_Z) may have the effect of ensuring that the method 802 does not reach the Tier 2 block 814. Alternatively, a low value for Th Z may cause the method 802 to more readily call for delivery Tier 2 therapy, which may occur with or without diaphragmatic stimulation. While the foregoing example mentions the threshold Th_Z, one or more other respiratory related criteria may be used (e.g., consider Th′ in the plot 863).

Another situation that may arise corresponds to pacing dependent patients. A pacing dependent patient generally requires continuous delivery of, for example, sinus pacing. Hence, for the decision block 824 and the input block 862, the input sinus rate may not be available or it may lack unreliable or un-actionable information. For example, in some patients, pacing may be halted for a short period of time to acquire an underlying intrinsic rate, however, this intrinsic beat may be sporadic and not useful for purposes of decision making. In such scenarios, the decision block 824 may be avoided. Alternatively, a different decision may occur based on a patient's current pacing rate and optionally on historical pacing rates (e.g., that may depend on other information such as patient activity). Further, where pacing rate for a pacing dependent patient relies on patient activity, such a relationship may be accounted for by the activity decision block 822. In such a scenario, the activity decision block 822 may proceed directly to the impedance decision block 826 when deciding whether a higher tier of therapy should be called for.

For some patients (e.g., an athlete), for a given level of activity, heart rate may not increase as much as breathing increases. For such patients, the heart rate threshold can be set to ensure that impedance (i.e., respiration) is accounted for when making a decision to call for a higher tier of therapy. Alternatively, the order of the heart rate decision block 824 and the respiration/impedance decision block 826 may be reversed to ensure that a higher tier of therapy can be called, if necessary.

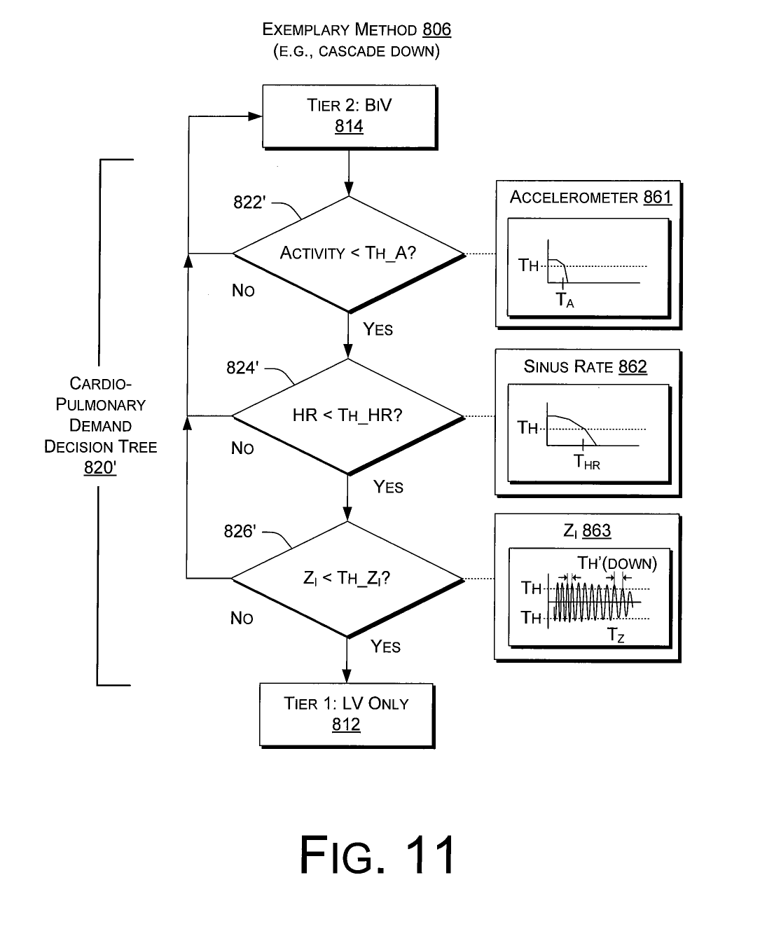

While the foregoing examples for diaphragmatic stimulation and a pacing dependent patient have been discussed with respect to the modules of FIG. 8 and the decision tree of FIG. 9, such examples may be optionally applied to the examples of FIGS. 11 through 14. Further, while the foregoing examples have been discussed with a move from a lower tier to a higher tier, similar techniques can be logically used when deciding whether to move from a higher tier to a lower tier.

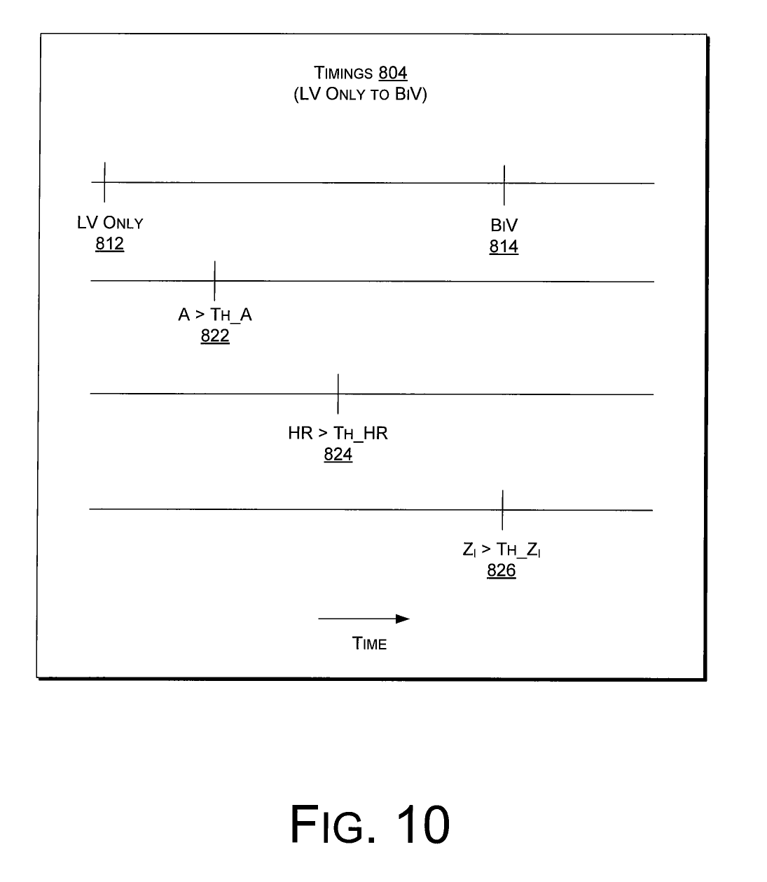

FIG. 10 shows timings 804 of the various events of the method 802. As already discussed, activity increases 822, followed by heart rate 824 and then respiratory rate or tidal volume 826. Once respiration (e.g., one or more respiratory characteristics) exceeds the corresponding threshold or thresholds, the method 802 selects BiV pacing 814.

FIG. 11 shows an exemplary method 806, which is referred to a downward cascade as the physiologic inputs are timed in a manner opposite when compared to the method 802 of FIG. 9. In FIG. 11, the decision tree 820′ (as opposed to decision tree 820) accounts for the fact that the higher tier 2 814 is being used and that the lower energy therapy tier 1 812 may be selected.

The method 806 commences in tier 2 therapy 814. The method 806 acquires information from the accelerometer 861. A corresponding plot shows activity versus time where activity falls below a threshold at a time T_{A}. The information is processed by the decision block 822′ of the decision tree 820′. If the time of the accelerometer reading is less than T_{A}, then the method 806 will continue delivery of tier 2 therapy 814. If the time is at or exceeding T_{A}, then the method 806 will acquire information as to sinus rate 862. A corresponding plot shows heart rate versus time where heart rate falls below a threshold at a time T_{HR}. The information is processed by the decision block 824′ of the decision tree 820′. If the time of the sinus rate reading is less than T_{HR}, then the method 804 will continue delivery of tier 2 therapy 814. If the time is at or exceeding T_{HR}, then the method 806 will acquire information as to respiration 863.

For respiration, a corresponding plot shows variation in impedance signal versus time where the signal exceeds a threshold at a time T_{Z}. A threshold may be for amplitude of impedance signal (which can indicate tidal volume) or peak-to-peak time difference (which can indicate respiration rate). The information is processed by the decision block 826′ of the decision tree 820′. If the time of the impedance reading is less than T_{Z}, then the method 806 will continue delivery of tier 2 therapy 814. Otherwise, the method 806 will select tier 1 LV therapy 812.

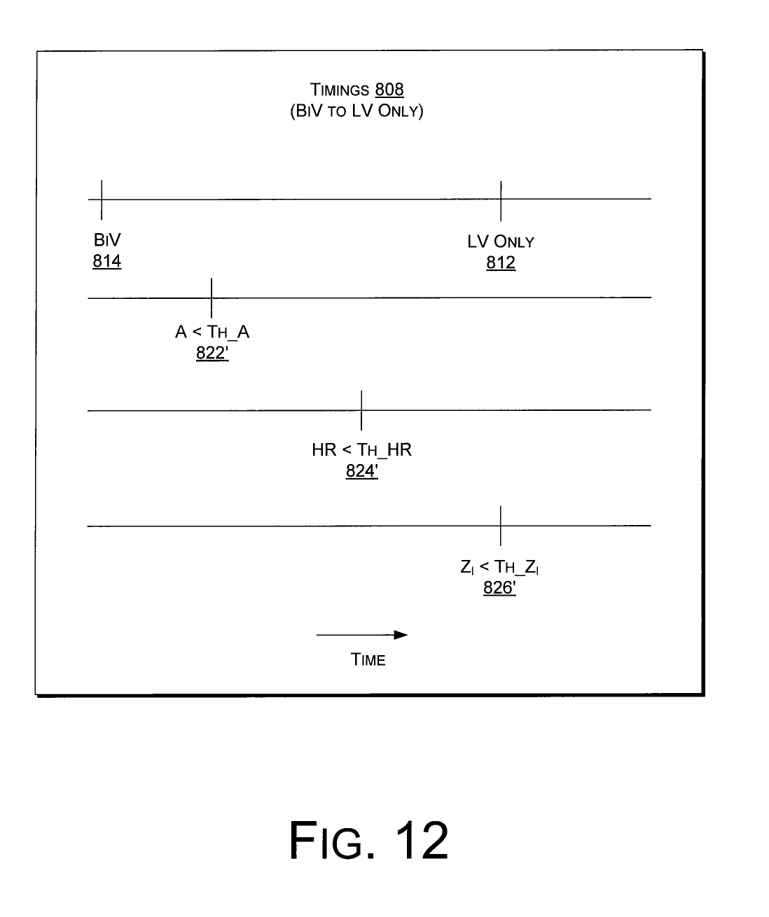

FIG. 12 shows timings 808 of the various events of the method 806. As already discussed, activity decreases 822′, followed by heart rate 824′ and then respiratory rate or tidal volume 826′. Once respiration (e.g., one or more respiratory characteristics) falls below the corresponding threshold or thresholds, the method 806 selects LV only pacing 812.

The timings 806 of FIG. 10 and 808 of FIG. 12 show occurrence of events for an increase in cardio-pulmonary demand and for a decrease in cardio-pulmonary demand, respectively. As described herein, a event that normally occurs prior to another event can be used to trigger sensing or acquisition of information. For example, an increase in activity may trigger sensing of heart rate and/or sensing of respiration and a decrease in activity may trigger sensing of heart rate and/or respiration.

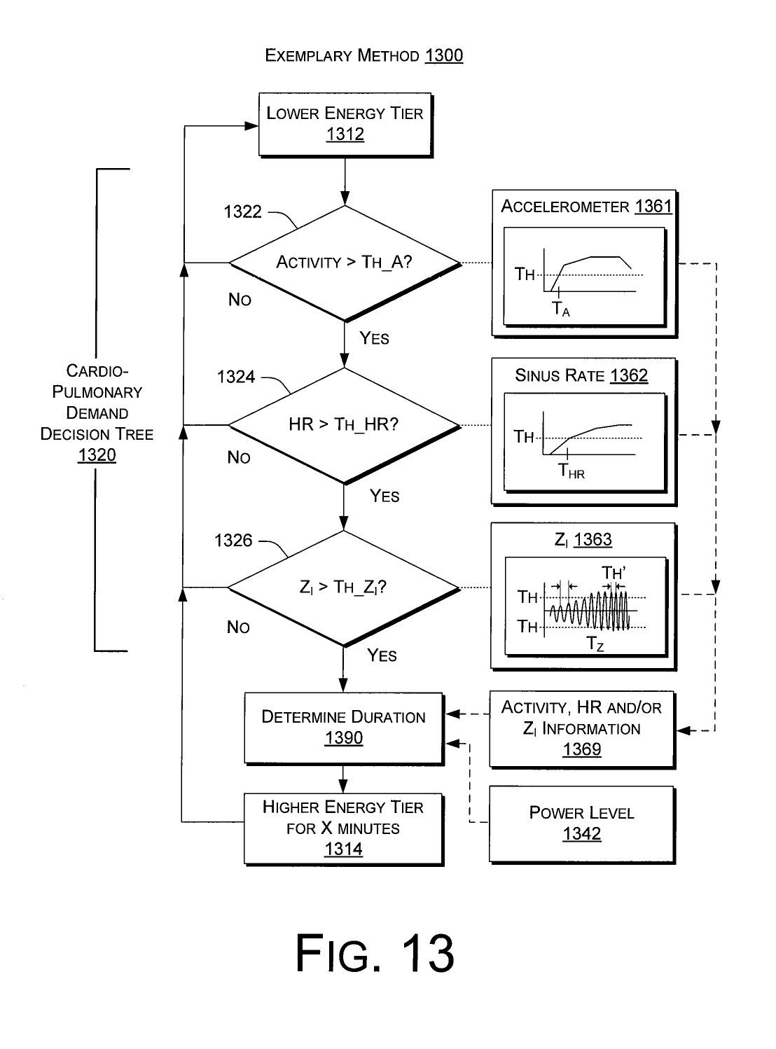

FIG. 13 shows an exemplary method 1300 for deciding whether to select a higher energy tier. The method 1300 commences in a delivery block 1312 that delivers a lower energy tier therapy. The method 1300 operates according to a cardio-pulmonary demand decision tree 1320, which may provide for decisions when demand increases, as shown, or when demand decreases. The decision making process may be based on thresholds or any of a variety of mechanisms to ultimately decide whether a different therapy should be selected for a given demand or demand trend.

As shown in the example of FIG. 13, the method 1300 acquires information from an accelerometer 1361 or other activity sensor for use in a decision block 1322 that decides if the patient activity exceeds a patient activity threshold Th_A. If the activity exceeds the threshold, then the method 1300 continues at another decision block along the cardio-pulmonary decision tree 1320; otherwise, the method 1300 continues at the delivery block 1312.

The next decision block 1324 relies on heart rate information and may acquire information from a heart rate sensor 1362 (e.g., IEGM or other sensor). The decision block 1324 decides if the heart rate exceeds a heart rate threshold Th_HR. If the activity exceeds the threshold, then the method 1300 continues at yet another decision block along the cardiopulmonary decision tree 1320; otherwise, the method 1300 continues at the delivery block 1312.

The next decision block 1326 relies on respiration information and may acquire information from an impedance measurement circuit 1363 (see, e.g., the circuit 278 of FIG. 2). The decision block 1326 decides if one or more characteristics of respiration indicate that cardio-pulmonary demand is, for example, above a threshold (e.g., impedance threshold Th_Z, a respiration rate, etc.). If the activity exceeds the threshold, then the method 1300 continues at a determination block 1390 that determines a duration for delivering a higher energy tier therapy 1314. For example, the determination block 1390 may receive activity, HR and/or impedance information 1369 and/or other information such as power level information 1342 and use received information to determine an appropriate duration for delivery of the higher energy tier 1314. Alternatively, for example, the duration may be pre-determined.

Once the method 1300 selects the higher energy tier 1314, it is delivered for the duration provided by the determination block. The example of FIG. 13 indicates that the higher energy tier 1314 is delivered for X minutes, where X represents the number of minutes a patient may be expected to need a boost in cardiac performance. After the time expires, the method 1300 returns to the delivery block 1312 where it selects the lower energy tier 1312.

While various examples refer to activity along with heart rate and respiration, other information may be used in deciding whether to select a different therapy tier. In general, for HF patients, characteristics of respiration reflect demand and can indicate whether a patient is adequately meeting the demand or whether a therapy should be selected to boost cardiac performance.

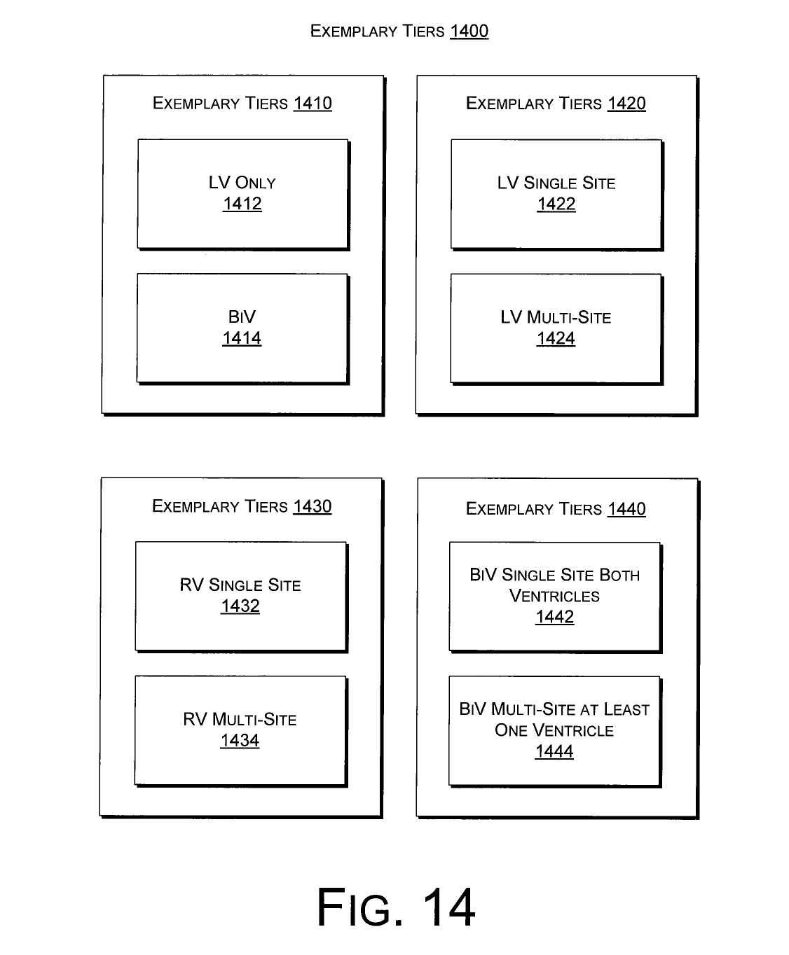

FIG. 14 shows exemplary tiers 1400, specifically tiers 1410, tiers 1420, tiers 1430 and tiers 1440. One or more of the tiers of FIG. 14, and/or one or more others described herein, may be provided via hardware and/or software (see, e.g., the device 100 of FIGS. 1 and 2). For example, a programmable device may include one or more modules with processor-executable instructions to cause the device to implement a pacing therapy according to a tier or tiers. A tier may also be referred to as a pacing algorithm. A decision tree, or decision algorithm, may be provided by hardware and/or software and operate to call for a delivery of therapy per a pacing tier in a multitier method (e.g., based on acquired information, a trigger, a timer, etc.).

In FIG. 14, the tiers 1410 include LV only 1412 and BiV pacing 1414. According to tiers 1410, if cardio-pulmonary demand increases, a pacing device (e.g., CRT device) can select the BiV pacing tier 1414; whereas for a decrease in demand, the pacing device can select or switch back to the LV pacing only tier 1412. The tiers 1420 include LV single site pacing 1422 and LV multi-site pacing 1424. According to tiers 1420, if cardio-pulmonary demand increases, a pacing device (e.g., CRT device) can select the pacing tier 1424; whereas for a decrease in demand, the pacing device can select or switch back to the pacing tier 1422. According to tiers 1430, if cardio-pulmonary demand increases, a pacing device (e.g., CRT device) can select the pacing tier 1434 (RV multi-site); whereas for a decrease in demand, the pacing device can select or switch back to the pacing tier 1432 (RV single site). According to tiers 1440, if cardiopulmonary demand increases, a pacing device (e.g., CRT device) can select the pacing tier 1444 (BiV multi-site in at least one ventricle); whereas for a decrease in demand, the pacing device can select or switch back to the pacing tier 1442 (BiV single site in both ventricles).

With a threshold set for activity level, the activity sensor can determine if a CRT patient is above or below the activity threshold. Any physiological parameters measurable by implanted sensor can be also determining factor for pacing mode selection or switching.

As described herein, an exemplary method for multi-tier pacing includes delivering single site, left ventricular pacing (see, e.g., block 404 of FIG. 4, block 1312 of FIG. 13 and block 1422 of FIG. 14); sensing patient activity (see, e.g., block 408 of FIG. 4 and block 1361 of FIG. 13); comparing the sensed patient activity to a patient activity threshold (see, e.g., block 412 of FIG. 4 and block 1322 of FIG. 13); if the sensed patient activity exceeds the patient activity threshold, then delivering multi-site, left ventricular pacing for a predetermined period of time (see, e.g., block 424 of FIG. 4, blocks 1314 and 1390 of FIG. 13 and block 1424 of FIG. 14); and after the predetermined period of time, delivering single, site left ventricular pacing (see, e.g., blocks 1314 and 1312 of FIG. 13). In this method for multi-tier pacing, the predetermined period of time may be a period of time based at least in part on cardiopulmonary demand and/or a period of time based at least in part on a battery level (see, e.g., blocks 1369 and 1342 of FIG. 13). Cardio-pulmonary demand may be based at least in part on the sensed patient activity (see, e.g., block 1369 of FIG. 13). In various examples, sensed patient activity is an indicator of cardio-pulmonary demand.

Another indicator of cardiopulmonary demand can be intrathoracic impedance, hence, an exemplary method may include sensing intrathoracic impedance (see, e.g., block 1363 of FIG. 13). Such sensing may occur during delivery of multi-site, left ventricular pacing. In various examples, impedance may be used to determine tidal volume. Further, an exemplary method may include comparing tidal volume to a tidal volume threshold and if the tidal volume exceeds the tidal volume threshold, then calling for delivery of multi-site, left ventricular pacing and right ventricular pacing. In an alternative, a method may include comparing the tidal volume to a tidal volume threshold and if the tidal volume exceeds the tidal volume threshold, then calling for delivery of multi-site, left ventricular pacing and multi-site, right ventricular pacing (see, e.g., block 1444 of FIG. 14). While calling for delivery of pacing is mentioned, various methods may include actually delivering the called for pacing (e.g., delivering multi-site, left ventricular pacing and multi-site right ventricular pacing for a predetermined period of time, etc.). Various exemplary methods may be implemented (in part or in whole) using a processor where one or more processor-readable media include processor-executable instructions.

As described herein, an exemplary method for multi-tier pacing includes providing a first pacing tier that includes left ventricular pacing only where the pacing paces the left ventricle at at least one left ventricular pacing site; providing a second pacing tier that includes bi-ventricular pacing; providing a third pacing tier that includes left ventricular pacing where the pacing paces the left ventricle at a greater number of left ventricular pacing sites than the first pacing tier; and selecting the first, second or third pacing tier for delivery of pacing therapy (see, e.g., FIG. 3 and FIG. 14 for various tiers).

As described herein, an exemplary method for multi-tier pacing includes providing a first pacing tier that includes single-site ventricular pacing; providing a second pacing tier that includes bi-ventricular pacing; providing a third pacing tier that includes multi-site ventricular pacing of a single ventricle; providing a decision tree to decide whether to select the first pacing tier, the second pacing tier or the third pacing tier based on cardio-pulmonary demand; and calling for delivery of pacing therapy according to the first pacing tier, the second pacing tier or the third pacing tier based on a decision of the decision tree (see, e.g., FIG. 3 and FIG. 14 for various tiers and decisions trees of FIGS. 4, 9, 11 and 13). Such a method may optionally include providing a fourth pacing tier that includes bi-ventricular pacing that further includes multi-site pacing in a single ventricle.

As described herein, a method for multi-tier pacing includes providing a first pacing tier that includes single-site ventricular pacing; providing a second pacing tier that includes bi-ventricular pacing; providing a third pacing tier that includes multi-site ventricular pacing of both ventricles; providing a decision tree to decide whether to select the first pacing tier, the second pacing tier or the third pacing tier based on cardiopulmonary demand; and calling for delivery of pacing therapy according to the first pacing tier, the second pacing tier or the third pacing tier based on a decision of the decision tree (see, e.g., FIG. 3 and FIG. 14 for various tiers and decisions trees of FIGS. 4, 9, 11 and 13).

As described herein, an exemplary method for improving hemodynamic performance includes delivering left ventricular pacing; detecting an increase in patient activity; in response to the detecting, delivering bi-ventricular pacing for a predetermined period of time; and after the predetermined period of time, delivering left ventricular pacing (see, e.g., the method 802 of FIG. 9 and block 1410 of FIG. 14). Such a method may include determining the predetermined period of time based at least in part on cardio-pulmonary demand and/or determining the predetermined period of time based at least in part on power supply level of an implantable device (see, e.g., blocks 1342 and 1369 of FIG. 13).

### Conclusion

Although exemplary methods, devices, systems, etc., have been described in language specific to structural features and/or methodological acts, it is to be understood that the subject matter defined in the appended claims is not necessarily limited to the specific features or acts described. Rather, the specific features and acts are disclosed as exemplary forms of implementing the claimed methods, devices, systems, etc.

What is claimed is:

1. A method for multi-tier pacing comprising:
delivering single site, left ventricular pacing;
sensing patient activity;
comparing the sensed patient activity to a patient activity threshold;
if the sensed patient activity exceeds the patient activity threshold, then delivering multi-site, left ventricular pacing for a predetermined period of time; and
after the predetermined period of time, delivering single site, left ventricular pacing.

2. The method of claim 1 wherein the predetermined period of time comprises a period of time based at least in part on cardio-pulmonary demand.

3. The method of claim 1 wherein the predetermined period of time comprises a period of time based at least in part on a battery level.

4. The method of claim 1 wherein the predetermined period of time comprises a period of time based at least in part on the sensed patient activity.

5. The method of claim 1 further comprising sensing intrathoracic impedance.

6. The method of claim 5 further comprising sensing intrathoracic impedance during the delivering multi-site, left ventricular pacing.

7. The method of claim 5 further comprising determining tidal volume based on the sensed intrathoracic impedance.

8. The method of claim 7 further comprising comparing the tidal volume to a tidal volume threshold and if the tidal volume exceeds the tidal volume threshold, then delivering multi-site, left ventricular pacing and right ventricular pacing.

9. The method of claim 7 further comprising comparing the tidal volume to a tidal volume threshold and if the tidal volume exceeds the tidal volume threshold, then delivering multi-site, left ventricular pacing and multi-site, right ventricular pacing.

10. The method of claim 9 further comprising delivering the multi-site, left ventricular pacing and multi-site right ventricular pacing for a predetermined period of time.

11. A method for multi-tier pacing, the method comprising:
providing a first pacing tier that comprises left ventricular pacing only wherein the pacing paces the left ventricle at at least one left ventricular pacing site;
providing a second pacing tier that comprises bi-ventricular pacing;
providing a third pacing tier that comprises left ventricular pacing wherein the pacing paces the left ventricle at a greater number of left ventricular pacing sites than the first pacing tier;
sensing patient activity; and
selecting the first, second or third pacing tier for delivery of pacing therapy based at least in part on the sensed patient activity.

12. A method for multi-tier pacing, the method comprising:
providing a first pacing tier that comprises single-site ventricular pacing;
providing a second pacing tier that comprises multi-site ventricular pacing;
providing a decision tree to decide whether to select the first pacing tier or the second pacing tier based on cardio-pulmonary demand; and
calling for delivery of pacing therapy according to the first pacing tier or the second pacing tier based on a decision of the decision tree.

13. The method of claim 12 wherein the decision tree comprises a decision based at least in part on patient activity.

14. The method of claim 12 wherein the decision tree comprises a decision based at least in part on heart rate.

15. The method of claim 12 wherein the decision tree comprises a decision based at least in part on respiration.

16. The method of claim 12 wherein the decision tree comprises at least one of a decision based at least in part on patient activity, a decision based at least in part on heart rate, and a decision based at least in part on respiration.

17. A method for multi-tier pacing, the method comprising:
providing a first pacing tier that comprises single-site ventricular pacing;
providing a second pacing tier that comprises bi-ventricular pacing;
providing a third pacing tier that comprises multi-site ventricular pacing of both ventricles;
providing a decision tree to decide whether to select the first pacing tier, the second pacing tier or the third pacing tier based on cardio-pulmonary demand; and
calling for delivery of pacing therapy according to the first pacing tier, the second pacing tier or the third pacing tier based on a decision of the decision tree.
